(19) United States
(12) Patent Application Publication
(10) Pub. No.: US 2026/0183356 A1
(43) Pub. Date: Jul. 2, 2026

(54) NOVEL COMBINATION BASED ON RIFAXIMIN AND PROBIOTIC YEASTS, COMPOSITIONS CONTAINING IT AND THEIR USE IN THERAPY

(71) Applicant: NYUMA PHARMA SRL, Arona (NO) (IT)

(72) Inventors: Angelo Pizzoni, Arona (NO) (IT); Paolo Pizzoni, Arona (NO) (IT)

(73) Assignee: NYUMA PHARMA SRL, Arona (NO) (IT)

(21) Appl. No.: 19/131,299

(22) PCT Filed: Nov. 28, 2023
(86) PCT No.: PCT/IB2023/061949

§ 371 Date: May 20, 2025

(30) Foreign Application Priority Data
Nov. 28, 2022 (IT) 102022000024489

(51) Int. Cl. A61K 36/064; A61K 9/20; A61K 31/437; A61P 1/00

(52) U.S. Cl. CPC A61K 36/064; A61K 9/2086; A61K 31/437; A61P 1/00

(57) ABSTRACT

The present invention relates to a novel combination of rifaximin and probiotic yeast, the pharmaceutical compositions containing the combination as well as their use in therapy, in particular in the prevention and/or treatment of intestinal diseases.

The present invention relates to a novel combination of rifaximin and probiotic yeast, the pharmaceutical compositions containing the combination as well as their use in therapy, in particular in the prevention and/or treatment of intestinal diseases.

### TECHNICAL BACKGROUND

Gastrointestinal infections cause stomach and intestinal disorders and symptoms include diarrhea, vomiting, and abdominal pain. Most gastrointestinal infections resolve within a few days; however, in particular individuals, such as young children, frail or elderly patients, these infections are potentially serious and may lead to dehydration.

There are various drugs on the market for the treatment of gastrointestinal infections, among which is rifaximin.

Rifaximin is an antibiotic that exerts its antibacterial activity in the gastrointestinal tract, against infections sustained by gram-positive and gram-negative bacteria that cause infectious diarrhea, irritable bowel syndrome, small intestinal bacterial overgrowth and Crohn's disease, but it is also used in the treatment of diverticular disease.

WO2015/047941 describes antibiotic-based compositions, including rifaximin, and probiotics for gastrointestinal disorders. In the compositions, antibiotics are formulated in a gastro-resistant layer and probiotics in an immediate-release layer. In paragraph of the description of WO2015/047941, it is reported that the composition allows a higher dose of antibiotics to be administered, and in the next paragraph ([0050]) it is mentioned that any antibiotic can be used. Finally, in paragraph it is reported that it is essential for the composition to release the antibiotic and probiotic at different sites and/or times in the gastrointestinal tract, because the antibiotic generally kills the probiotic bacteria as well (paragraph [0097]).

It is known that during the antibiotic treatment, gastrointestinal infections would also benefit from the administration of probiotic agents, and the combination of antibiotics and probiotics would theoretically represent a powerful synergistic therapy. Unfortunately, as reported also in WO2015/047941, most probiotic agents are antibiotic-sensitive and their action is therefore nullified by concurrent administration of antibiotics.

Therefore, there is a need to have novel therapeutic approaches that take advantage of combinations of active agents in order to more comprehensively treat gastrointestinal infections, thus acting through different mechanisms of action but avoiding negative interference between the components of the administered combinations.

### OBJECTS OF THE INVENTION

A first object of the present invention is to provide a novel combination of rifaximin with probiotic yeast and its use in therapy, in particular in the prevention and/or treatment of gastrointestinal infections, their complications and diverticular disease.

Another object of the present invention is to provide pharmaceutical compositions containing the combination of the invention and their use in therapy, in particular in the prevention and/or treatment of gastrointestinal infections, their complications and diverticular disease.

### DESCRIPTION OF THE INVENTION

Subject-matter of the invention, according to one of its aspects, is a combination consisting of rifaximin and at least one probiotic yeast selected from the species of the Kluyveromyces genus.

Rifaximin is the International Nonproprietary Name for a known antibiotic belonging to the rifamycin group. Systemic absorption of rifaximin is negligible and it is widely used in the therapy of gastrointestinal infections, their complications and diverticular disease. It is commercially available, for example under the trade names Normix® and Rifacol®.

According to the invention, the at least one probiotic yeast selected from the species of the Kluyveromyces genus is preferably selected from a Kluyveromyces marxianus species.

According to a preferred embodiment, said at least one yeast is the Kluyveromyces marxianus B0399 strain.

Yeasts according to the invention are known and commercially available.

According to a preferred embodiment, said at least one yeast is in lyophilized form.

According to a preferred embodiment, the combination of the invention is constituted by rifaximin and the Kluyveromyces marxianus B0399 yeast.

Completely unexpectedly, the Applicant was able to demonstrate that the yeast Kluyveromyces marxianus is surprisingly able to resist the antibiotic action of rifaximin, in contrast to pathogenic and probiotic bacteria. Even more surprisingly, its growth is instead inhibited by the antibiotic rifamycin, despite having a very similar molecular structure to that of rifaximin and sharing its mechanism of action.

Therefore, it is understood that the combination of rifaximin and Kluyveromyces marxianus B0399 is an unexpected, novel and powerful therapy of gastrointestinal infections, their complications and diverticular disease, as will be discussed below.

For its administration to a subject in the need thereof, the combination of the invention is preferably formulated in a pharmaceutical composition.

Subject-matter of the invention, according to another of its aspects, is a pharmaceutical composition comprising the combination of the invention, in all of its forms set forth herein, and possibly one or more pharmaceutically acceptable excipients.

The composition of the invention may also comprise other active ingredients useful in the treatment and/or prevention of gastrointestinal conditions.

By way of example, the composition of the invention may comprise a pharmaceutically/physiologically acceptable salt of butyric acid, preferably the sodium salt.

Said pharmaceutically/physiologically acceptable salt of butyric acid, preferably the sodium salt, is advantageously in the (micro) encapsulated form.

According to a preferred embodiment, the composition of the invention comprises rifaximin, yeast of the Kluyveromyces marxianus species, preferably the Kluyveromyces marxianus B0399 strain, and a pharmaceutically/physiologically acceptable salt of butyric acid, preferably the sodium salt.

According to a preferred embodiment, the composition of the invention is suitable for the oral administration.

According to a preferred embodiment, the combination is contained in solid oral compositions, possibly to be diluted with a liquid.

According to an embodiment, the combination is contained in solid oral compositions of the capsule, sachet or tablet type, preferably a tablet or granulate.

According to a preferred embodiment, the combination is contained in oral solid dosage units of the bilayer tablet type.

The bilayer tablets are known in the art and have the advantage of allowing two active ingredients to be administered at the same time, without having to mix them.

Any one of the methods known in the art may be used to prepare the bilayer compositions of the invention. The two layers may be of the same or different colors.

The two layers of the bilayer composition may contain any pharmaceutically acceptable excipient and/or carrier which are suitable and conventionally used in the preparation of said compositions, e.g., diluents, bulking agents, binders, disaggregants, flow aids, lubricants, etc. Non-limiting examples of suitable carriers and excipients are described in “Remington: The Science and Practice of Pharmacy, 21° Ed., Lippincott, Williams & Wilkins”. The compositions of the invention may include, for example, cellulose derivatives, glucose, lactose, sucrose, gelatin, malt, rice, flour, gypsum, silica gel, sodium stearate, glycerol monostearate, starch, silica, talc, microcrystalline cellulose, hydroxypropyl methyl cellulose, sodium chloride, dyes, preservatives and the like.

The two components of the combination of the invention are included, each separately, in one of the two layers of the bilayer tablet.

The oral solid composition of the invention, preferably in the form of bilayer tablet, may comprise 100 to 500 mg rifaximin, preferably 150 to 300 mg, even more preferably about 200 mg rifaximin.

The oral solid composition of the invention, preferably in the form of bilayer tablet, may comprise 5 to 15 million CFU (colony-forming units) of at least one yeast of the species of the Kluyveromyces marxianus genus, preferably of the Kluyveromyces marxianus species, more preferably the Kluyveromyces marxianus B0399 strain, more preferably about 10 million CFU.

The oral solid composition of the invention is preferably in the form of bilayer tablet in which a layer comprises 100 to 500 mg rifaximin, preferably 150 to 300 mg, even more preferably about 200 mg rifaximin and the other layer comprises 5 to 15 million CFU (colony-forming units) of the species of the Kluyveromyces genus, preferably the Kluyveromyces marxianus species, more preferably the Kluyveromyces marxianus B0399 strain, more preferably about 10 million CFU.

Preferably the yeast according to the invention, preferably the Kluyveromyces marxianus, more preferably the Kluyveromyces marxianus B0399 strain, is in lyophilized form.

When a pharmaceutically/physiologically acceptable salt of butyric acid, preferably the sodium salt, is also present in the composition, such compound may be included in the tablet layer containing rifaximin, especially when it is in the (micro) encapsulated form.

Alternatively, when a pharmaceutically/physiologically acceptable salt of butyric acid, preferably the sodium salt, is also present, such compound is preferably present in a separate layer and the resulting tablet is thus a “tri-layer” tablet.

When present, said pharmaceutically/physiologically acceptable salt of butyric acid, preferably the sodium salt, is contained in the composition of the invention at the rate of 100 to 400 mg, preferably about 200 mg, per dosage unit.

According to an alternative embodiment, the combination is contained in two separate pharmaceutical compositions, which are mixed before ingestion.

According to a preferred embodiment, rifaximin is in the form of granulate and the Kluyveromyces marxianus yeast, preferably the Kluyveromyces marxianus B0399 strain, is in lyophilized form.

According to the alternative “single-dose” (or dosage unit) embodiment, the rifaximin granulate is contained in a bottle with a storage cap that in turn contains said yeast, preferably in lyophilized form, both in an amount suitable for one administration (dosage unit).

At the time of the administration, the rifaximin granulate and the yeast are mixed and water is added so as to form a suspension, which is then taken as a dosage unit.

Such dosage unit comprises 100 to 500 mg rifaximin, preferably 150 to 300 mg, even more preferably about 200 mg rifaximin and 5 to 15 million CFU (colony-forming units) of Kluyveromyces marxianus, preferably the Kluyveromyces marxianus B0399 strain, more preferably about 10 million CFU.

According to the alternative “multi-dose” embodiment, the bottle contains multiple doses of rifaximin granulate and the storage cap, in turn, contains the equivalent of multiple doses of said yeast, preferably in lyophilized form.

The bottle provided with a multi-dose storage cap will also be provided with a meter for taking the correct dosage.

According to a preferred embodiment, the bottles (single-dose and multi-dose) are provided with indicators for the level of water to be added. Preferably, water is added until a suspension containing 100 mg rifaximin in 5 ml is obtained.

Thus, for example, for the administration of 200 mg rifaximin, 10 ml suspension should be taken.

When the said pharmaceutically/physiologically acceptable salt of butyric acid, preferably the sodium salt and preferably in the (micro) encapsulated form, is also present in the composition according to the aforementioned alternative embodiment, this compound is contained in the bottle together with rifaximin.

The compositions of the invention are preferably packaged with an accompanying leaflet.

Subject-matter of the invention, according to another of its aspects, is rifaximin for its use in the treatment and prevention of gastrointestinal infections, their complications and diverticular disease, in combination with probiotic yeast selected from the Kluyveromyces genus, preferably selected from Kluyveromyces marxianus, more preferably with the probiotic yeast of the Kluyveromyces marxianus B0399 strain.

For use in the combination of the invention, rifaximin and said yeast may be administered simultaneously or sequentially, within a short time frame, so that they may exert contextually their action in the body. Preferably, rifaximin and said yeast are taken simultaneously, or at most within a time frame of one or two hours.

For their administration according to the combined use of the invention, rifaximin and said yeast may be formulated in conventional pharmaceutical compositions.

The compositions of the invention are administered once or more times a day and the dosages can vary according to the weight and age of the subject to be treated, as well as the type and severity of the disease.

The “subject” according to the invention is a mammal, preferably, but not only, a human being.

Subject-matter of the invention, according to another of its aspects, is the combination of the invention for its use in therapy, in particular in the treatment and/or prevention of gastrointestinal infections, their complications and diverticular disease.

The compositions of the invention for their use in therapy, in particular in the treatment and/or prevention of the gastrointestinal infections, their complications and diverticular disease, are subject-matter of the invention according to another of its aspects.

Subject-matter of the invention, according to another of its aspects, is a method for the treatment and/or prevention of gastrointestinal infections, their complications and diverticular disease, which comprises administering an effective amount of the combination or compositions of the invention to a subject in the need thereof.

In particular, the combination and compositions of the invention are particularly suitable for the treatment of acute and chronic intestinal infections sustained by gram-positive and gram-negative bacteria, diarrheal syndromes and diarrhea due to altered balance of intestinal microbial flora, such as summer diarrhea, traveler's diarrhea and enterocolitis.

The combination and compositions of the invention are also useful in pre- and post-operative prophylaxis of infectious complications in gastrointestinal tract surgeries.

The combination and compositions of the invention are also for example useful in the treatment of the irritable bowel syndrome of Crohn's disease and the like.

Furthermore, the combination and compositions of the invention are particularly suitable for the prevention and/or treatment of diverticular disease.

Diverticular disease (MD) is a clinical condition, the prevalence of which (age-dependent) is continuously increasing. Along with a symptomatology (abdominal pain and swelling, diarrhea and/or constipation) that overlaps with that of the Irritable Bowel Syndrome (IBS), MD may lead to inflammation/infection of the diverticula, resulting in a clinical picture of acute diverticulitis, which may be complicated or uncomplicated, the management of which is particularly difficult especially in the elderly patient.

Dysbiosis (and in particular the Small Intestine Bacterial Overgrowth-SIBO) is an alteration in intestinal micro-ecology which is frequently associated with diverticular disease. Among the available therapies for dysbiosis, rifaximin is the most widely used antibiotic, as it is extremely active on the dysbiosis and is also able to increase the relative abundance of Lactobacilli, Bifidobacteria and Fecalibacterium prausnitzi, an endogenous probiotic with anti-inflammatory activity. The probiotic agents could further improve the clinical picture if they are combined with rifaximin in the treatment of dysbiosis, because they are able to favorably modify the microbiota. Unfortunately, as mentioned, most probiotics are antibiotic-sensitive and are therefore are destroyed by the concurrent administration of antibiotics, including rifaximin.

Therefore, it is understood that the probiotic yeast, as defined here, due to its inherent resistance to antibiotics, allows realizing a particularly effective combination against gastrointestinal infections and MD, because such a combination acts on two fronts and thus provides better results than the use of rifaximin alone.

Subject-matter of the invention, according to another of its aspects, is a kit which comprises pharmaceutical compositions containing rifaximin and pharmaceutical compositions containing at least one probiotic yeast selected from the species of Kluyveromyces genus, preferably selected from the Kluyveromyces marxianus species, more preferably with the probiotic yeast of the Kluyveromyces marxianus B0399 strain.

According to an embodiment, the kit of the invention comprises 1 to 100 pharmaceutical compositions containing rifaximin and 1 to 100 pharmaceutical compositions containing at least one probiotic yeast, e.g., 7 to 50, preferably 10 to 30 of each composition, the number of pharmaceutical compositions containing rifaximin and the number of pharmaceutical compositions containing at least one probiotic yeast being preferably the same.

The compositions contained in the kit of the invention can contain suitable pharmaceutically acceptable excipients and carriers.

The preferred embodiments described above for the combination and compositions containing the combination also apply to the compositions of the kit.

The compositions of the kit can be taken simultaneously or otherwise within a short time frame, as set forth above.

The compositions of the kit are preferably packaged together with a leaflet.

The invention will be now described in more detail in the Experimental Section below, by way of illustration only and in no way limiting.

### EXPERIMENTAL SECTION

### Example 1

A composition is prepared in the form of bilayer tablet for oral use having
a layer containing 10 million CFU of Kluyveromyces marxianus B0399 in lyophilized form;
a layer containing 200 mg rifaximin;
together with pharmaceutically acceptable excipients and/or carriers.

### Example 2

A composition in the form of granulate for oral use, together with pharmaceutically acceptable excipients and/or carriers, is prepared and an amount thereof containing 200 mg rifaximin is added into a bottle provided with an indicator for the level of water to be added; said bottle is capped with a storage cap containing 10 million CFU of Kluyveromyces marxianus B0399 in lyophilized form.

### Example 3

A composition is prepared in the form of tri-layer tablet for oral use having
a layer containing 10 million CFU of Kluyveromyces marxianus B0399 in lyophilized form;
a layer containing 200 mg rifaximin; and
a layer containing 200 mg sodium salt of (micro) encapsulated butyric acid;
together with pharmaceutically acceptable excipients and/or carriers.

### Example 4

A composition is prepared in the form of bilayer tablet for oral use having
a layer containing 10 million CFU of Kluyveromyces marxianus B0399 in lyophilized form;
a layer containing 200 mg rifaximin and 200 mg sodium salt of (micro) encapsulated butyric acid;
together with pharmaceutically acceptable excipients and/or carriers.

### Example 5

Determination of Susceptibility of Kluyveromyces marxianus B099 to the Antibiotic Rifaximin

The test aims to assess the in vitro susceptibility of the yeast Kluyveromyces marxianus to the antibiotic rifaximin. The test is performed by assaying 8 scalar dilutions of rifaximin in liquid culture medium (microdilution). Bacterial strains are tested in parallel as controls. Rifaximin is a synthetic molecule derived from rifamycin. The powder is characterized by poor solubility in water and tendency to self-aggregate in the liquid phase. A preliminary step is then performed to set the solubility conditions of rifaximin at the highest concentration used in the test (0.5 mg/ml).

The following strains were used
Kluyveromyces marxianus B099
Bifidobacterium longum W11
Escherichia coli ATCC 8739

The Escherichia coli bacterial strain was revitalized from a reference stock suspension stored at −80° C. After thawing, working cultures were set up by growing the strain in suitable plate medium under the optimal growth conditions. The other strains were obtained after plating on agarized culture medium of the specific matrices and subsequent isolation of pure colonies. In Table 1 the strains and the growth and inoculation conditions are set forth.

TABLE 1

| | | Starting |
|---|---|---|
| | | inoculum |
| STRAIN | Growth conditions | (CFU/ml) |
| Escherichia coli | Tryptic soy broth Tryptic soy | 3.2 × 10³ |
| ATCC 8739 | agar (1-2 days - 37° C.) | |
| Kluyveromyces marxianus | Sabouraud dextrose broth | 5.6 × 10³ |
| fragilis B0399 | Sabouraud dextrose | |
| | agar (2-3 days - 37° C.) | |
| Bifidobacterium longum | Tryptic soy broth Anaerobe | 1.1 × 10³ |
| W11 | basal agar + Defibrinated | |
| | Sheep Blood | |
| | (2-3 days - 37° C. anaerobiosis) | |

### Preliminary Feasibility Test

The exploratory step is performed to set the solubilization conditions of rifaximin and the culture conditions of the microbial strains. Given the poor solubility of rifaximin powder in water and the tendency for self-aggregation in the liquid phase, a solubilization test in DMSO (dimethyl sulfoxide) is performed. From the preliminary solubilization tests, the 50 mg/ml concentration was found to be the highest with no visible precipitates and clear, thus compatible with performing the test (starting stock solution). The first concentration used in the test and the subsequent dilutions (set up in Tryptic soy broth-TSB or Sabouraud dextrose broth-SDB, base-2 serial dilutions) were defined in agreement with the client, based on literature data. The strains were expanded first in liquid medium and then in agarized medium, as described in Table 2.2.1, obtaining growth results that confirmed the use of these media and conditions for the microdilution test as well.

### Microdilution Test

In the microdilution test, broth-cultures of the test microorganisms are set up in triplicates in the presence of 8 different concentrations of rifaximin, which are selected in agreement with the client, and 1 concentration of rifamycin corresponding to the highest concentration selected for rifaximin. 100 μl of a solution, 2 times more concentrated (2×) than the desired concentration, and 100 μl of the microbial suspension at a titer given in Table 2.2.1, were added to each well of the 96-wells plates. The 8 concentrations of rifaximin are produced from the 50 mg/ml solution in DMSO in TSB and in parallel in SDB.

The final test concentrations are as follows: 0.5 mg/ml-0.25 mg/ml-0.125 mg/ml-0.063 mg/ml-0.031 mg/ml-0.015 mg/ml-0.0078 mg/ml-0.0039 mg/ml.

### Result Interpretation

Following the absorbance measurement on the spectrophotometer, the mean of the data recorded for each concentration and the mean of the data from the controls in DMSO or sterile water are calculated. To estimate the microbial growth and determine MIC from the mean data obtained (in the test and controls), the blank data are subtracted and then the control data and the test data are compared with each other to assess any growth or growth inhibition of each strain tested. The growth inhibition is calculated as a percentage by the following formula:

$$Inhibition ( \% ) = 100 - ( ( B / A ) * 100 )$$

wherein
A: Mean OD of control
B: Mean OD of the sample at the concentration tested
MIC is considered the lowest concentration of product that can give at least 90% growth inhibition.

The results are shown in Table 2.

### Table 2

Abatement of Strain Growth with Rifaximin

| | Concentration tested | | | | | | | |
|---|---|---|---|---|---|---|---|---|
| | 0.5 | 0.25 | 0.125 | 0.063 | 0.031 | 0.015 | 0.0078 | 0.0039 |
| | mg/ml | mg/ml | mg/ml | mg/ml | mg/ml | mg/ml | mg/ml | mg/ml |
| Kluyveromyces | −4.7% | −1.2% | −3.5% | −2.6% | −2.1% | −3.3% | −1.7% | 1.5% |
| marxianus | | | | | | | | |
| Escherichia coli | 98.1% | 98.5% | >99.9% | 99.6% | >99.9% | 94.5% | 54.9% | 45.7% |
| Bifidobacterium | >99.9% | 98.7% | >99.9% | >99.9% | >99.9% | >99.9% | >99.9% | 45.5% |
| longum W11 | | | | | | | | |

From the data of percentage growth abatement, it is clear that the Kluyveromyces marxianus strain is not abated and its growth is comparable to the growth of the control.

Escherichia coli strain is inhibited by rifaximin in amounts greater than or close to 99% at ≥0.031 mg/ml concentrations. Bifidobacterium longum strain is inhibited in >99.9% at virtually all concentrations except at 0.0039 mg/ml.

### Conclusions

The results demonstrate that rifaximin inhibits the growth of the pathogen Escherichia coli and also of the probiotic Bifidobacterium longum, whereas it does not inhibit the growth of the yeast Kluyveromyces marxianus.

### Example 6

Determination of Susceptibility of Kluyveromyces marxianus B099 to the Antibiotic Rifamycin

The strains in Example 5 were also tested to the antibiotic rifamycin, having a structure very similar to rifaximin.

A solution of rifamycin is prepared, at the 50 mg/ml concentration in sterile water, which is then diluted in tryptic soy broth (TSB) and in parallel in Sabouraud dextrose broth (SDB) until the 0.5 mg/ml concentration used in the test is obtained. The plates are incubated at the optimal temperatures and growth conditions of the various strains tested as described in Example 5. After the days of incubation have passed, the absorbance at 600 nm (OD600) is measured by spectrophotometer. The mean values after subtracting the blank are used to estimate microbial growth and determine MIC.

The results are shown in Table 3.

### Table 3

Abatement of Strain Growth with Rifamycin

| | Concentration tested 0.5 mg/ml |
|---|---|
| Kluyveromyces marxianus | 88.9% |
| Escherichia coli | 79.7% |
| Bifidobacterium longum W11 | 87.5% |

### Conclusions

The results demonstrate that, in contrast to rifaximin, rifamycin inhibits the growth of all three strains, including the yeast Kluyveromyces marxianus.

1. A combination consisting of rifaximin and at least one probiotic yeast selected from the species of the Kluyveromyces genus.

2. The combination according to claim 1, characterized in that said at least one yeast is selected from the species Kluyveromyces marxianus.

3. The combination according to claim 2, characterized in that said at least one yeast is the Kluyveromyces marxianus B0399 strain.

4. The combination according to any one of claims 1 to 3, characterized in that said yeast is in lyophilized form.

5. A pharmaceutical composition comprising the combination according to any one of claims 1 to 4, together with one or more pharmaceutically acceptable carriers and/or excipients.

6. The composition according to claim 5, characterized in that it is for oral administration.

7. The composition according to claim 6, characterized in that it is in the form of tablet.

8. The composition according to claim 7, characterized in that it is in the form of bilayer tablet.

9. The composition according to claim 6, characterized in that rifaximin is in the form of granulate, said granulate being contained in a bottle provided with a storage cap, said storage cap containing said yeast in lyophilized form.

10. The composition according to any one of claims 5 to 9, also comprising a pharmaceutically/physiologically acceptable salt of butyric acid, preferably the sodium salt, preferably in (micro) encapsulated form.

11. The combination according to any one of claims 1 to 4 or the composition according to any one of claims 5 to 10, for its use in therapy, preferably in the treatment and/or prevention of gastrointestinal infections, their complications and diverticular disease.

12. A kit comprising pharmaceutical compositions containing rifaximin, and compositions containing at least one probiotic yeast selected from the Kluyveromyces species, preferably selected from the Kluyveromyces marxianus species, more preferably with the Kluyveromyces marxianus B0399 strain.

13. The kit according to claim 12, characterized in that said yeast is in lyophilized form.

14. Rifaximin for its use in the treatment and prevention of the gastrointestinal infections, their complications and diverticular disease, in combination with at least one probiotic yeast selected from the Kluyveromyces species, preferably selected from the Kluyveromyces marxianus species, more preferably with the Kluyveromyces marxianus B0399 strain.
